(19) United States
(12) Patent Application Publication
(10) Pub. No.: US 2024/0189139 A1
(43) Pub. Date: Jun. 13, 2024

(54) COMPOSTABLE AERATOR URINE COLLECTOR

(71) Applicant: WSA Inventions, LLC, Sheridan, WY (US)

(72) Inventor: Peter NEWELL, Warner Springs, CA (US)

(73) Assignee: WSA Inventions, LLC, Sheridan, WY (US)

(21) Appl. No.: 18/530,664

(22) Filed: Dec. 6, 2023

Related U.S. Application Data
(60) Provisional application No. 63/386,461, filed on Dec. 7, 2022.

(51) Int. Cl. A61F 5/453; A61F 5/44

(52) U.S. Cl. CPC A61F 5/453; A61F 5/4401; A61F 5/4408; A61M 2202/0496

(57) ABSTRACT

A urine collection device and urine collection apparatus are disclosed. The urine collection device includes an aerator, which may include a channel may include paper, a first fabric layer covering an inner circumference of the channel, a second fabric layer covering an outer circumference of the channel, a retention ring at a proximal end of the aerator, attached to the first fabric layer and the second fabric layer, and a first connection ring at a distal end of the aerator, attached to the first fabric layer and the second fabric layer. The device also includes a receptacle, which may include a second connection ring at a proximal end of the receptacle, attached to the receptacle. The device can also include an absorbent media held within the receptacle. The urine collection apparatus includes a garment which includes an external coupling to which the urine collection device may be fastened.

### REFERENCE TO RELATED APPLICATIONS

This application claims the benefit of U.S. Provisional Patent Application No. 63/386,461, filed on Dec. 7, 2022, which is hereby incorporated by reference in its entirety.

### TECHNICAL FIELD

The present teachings relate generally to biodegradable medical accessories and, more particularly, to a compostable urine collector.

### BACKGROUND

Disposable absorbent products are widely used in the fields of health care, personal hygiene, medicine, pet care, and in any manner where a material or devices used for the absorption or collection of liquids and/or bodily fluids may be needed. Health and household items such as urinary incontinence devices, condom catheters, disposable diapers, and the like typically have an absorbent core material made using absorbent gels or polymers that may not be supportive of eco-friendly or environmental conscious goals. The presence of some of these absorbent constituents and other materials prevents these products from being recycled or from being used comfortably or hygienically. Existing products can lead to situations of prolonged contact of bodily fluids with the skin of the user, may promote bacterial, yeast, or fungal growth, discomfort during use, and conditions where they are typically disposed of in a landfill where it can take years for them to degrade. Not only do the products take a long time to degrade, the amount of trash that is accumulated from used diapers and the like is substantial. The waste created sits in landfills after disposal and potentially adding dangerous microplastics to the environment.

While devices incorporating externally used condom catheters showed limited improvements in comfort, the device requires direct attachment with adhesive to secure the condom to the penis. These devices are not comfortable during longer term use and may be conducive to minor penile injury via moisture, fungus, yeast, bacteria, reactions with the adhesive used, or a combination thereof. Diaper-like personal care garments can be bulky and unpleasant during long term use, particularly with a prolonged skin contact with urine by the wearer.

Therefore, it is desirable to provide a hygienic, comfortable, and easy to use collection device for urine, that is disposable and even home compostable, in this case for male patients for everyday convenience for incontinence, hospitalization, or other periods of immobility or restricted access to appropriate facilities.

### SUMMARY

The following presents a simplified summary in order to provide a basic understanding of some aspects of one or more embodiments of the present teachings. This summary is not an extensive overview, nor is it intended to identify key or critical elements of the present teachings, nor to delineate the scope of the disclosure. Rather, its primary purpose is merely to present one or more concepts in simplified form as a prelude to the detailed description presented later.

A urine collection device is disclosed. The urine collection device includes an aerator, which may include a channel may include paper, a first fabric layer covering an inner circumference of the channel, a second fabric layer covering an outer circumference of the channel, a retention ring at a proximal end of the aerator, attached to the first fabric layer and the second fabric layer, and a first connection ring at a distal end of the aerator, attached to the first fabric layer and the second fabric layer. The device also includes a receptacle, which may include a second connection ring at a proximal end of the receptacle, attached to the receptacle. The device can also include an absorbent media held within the receptacle.

Implementations of the urine collection device may include where the retention ring is configured to be reversibly fastened to a garment. The first connection ring and the second connection ring can be configured to reversibly fasten the aerator to the receptacle. The first connection ring may include one or more prominences on an outer circumference. The second connection ring can include one or more recesses on an inner circumference. The one or more prominences on the inner circumference of the first connection ring correspond to and interlock with the one or more recesses on the inner circumference of the second connection ring to fasten the aerator to the receptacle. The receptacle may include a liquid proof material. The absorbent media may include a biodegradable material, a fabric material enclosure, and a biodegradable fill material, and biodegradable waterproof covering. The absorbent media may include cotton, bamboo fluff, or other readily biodegradable material. The channel may include a segmented channel which may include more than one segment. The segmented channel may include five segments. The channel may include a plurality of perforations, a corrugated material, or a combination thereof. The first fabric layer may include a non-woven needle perforated (NWNP) fabric of bamboo or other biodegradable material. The second fabric layer may include a non-woven fabric, a needle penetrated fabric, or a combination thereof.

A urine collection apparatus is also disclosed. The urine collection apparatus includes a garment which includes an external coupling. The apparatus also includes an aerator, which may include a channel that can include paper, a first fabric layer covering an inner circumference of the channel, a second fabric layer covering an outer circumference of the channel, a retention ring at a proximal end of the aerator, attached to the first fabric layer and the second fabric layer, and a first connection ring at a distal end of the aerator, attached to the first fabric layer and the second fabric layer. The apparatus also includes a receptacle, including a second connection ring at a proximal end of the receptacle, attached to the receptacle, and an absorbent media held within the receptacle.

Implementations of the urine collection apparatus may include where the retention ring is configured to be reversibly fastened to the external coupling on the garment. The first connection ring and the second connection ring are configured to reversibly fasten the aerator to the receptacle. The aerator further may include a flexible portion at a distal end. The aerator further may include a flexible portion at a proximal end. The garment may include a pouch to retain the receptacle or at least a portion of the aerator or the receptacle. The pouch is included in one or more leggings of the garment. The pouch is internal to the garment.

The features, functions, and advantages that have been discussed can be achieved independently in various implementations or can be combined in yet other implementations further details of which can be seen with reference to the following description.

### BRIEF DESCRIPTION OF THE DRAWINGS

The accompanying drawings, which are incorporated in and constitute a part of this specification, illustrate embodiments of the present teachings and together with the description, serve to explain the principles of the disclosure. In the figures:

FIG. 1 is a schematic view of an aerating urine collection article, in accordance with the present disclosure.

FIG. 2 is a schematic view of an aerating urine collection apparatus, in accordance with the present disclosure.

FIG. 3 is a schematic view of an aerating urine collection apparatus, in accordance with the present disclosure.

It should be noted that some details of the figures have been simplified and are drawn to facilitate understanding of the present teachings rather than to maintain strict structural accuracy, detail, and scale.

### DETAILED DESCRIPTION

Reference will now be made in detail to exemplary embodiments of the present teachings, examples of which are illustrated in the accompanying drawings. Wherever possible, the same reference numbers will be used throughout the drawings to refer to the same, similar, or like parts.

If not specifically stated herein, any percent or amount by weight is by weight of total solids or the specific layer, part, or material of article being described.

The term “compostable” means any material that will decompose naturally as a result of the action of aerobic bacteria, fungi, and other organisms without leaving synthetic and potentially harmful material in the soil.

The term “biodegradable” means capable of being destroyed and broken down into very small parts by natural processes, bacteria, fungi or other natural organisms or means.

The term “bio-based” material is a material intentionally made from or derived from substances derived from living (or once-living) organisms.

The term “bodily fluids” refers to any fluid material naturally produced by animals including humans, and includes blood, discharge, urine, saliva, and similar bodily fluids.

Dimensions such as length, thickness, etc. may refer to an average value by which it is meant an average of the values from members of a population, for example, average length determined from a population of fibers, or average of the values from multiple locations, such as average thickness determined from multiple locations across the surface of a layer. In other instances, a thickness may refer to a value determined at a particular location, such as the center of an absorbent article.

Use of directional terms, such as distal, proximal, top, bottom, right, left, front, back, upper, lower, etc. are merely intended to facilitate reference to various surfaces that form components of the absorbent articles referenced herein and are not intended to be limiting in any manner.

The absorbent article herein can be used to absorb most liquid materials, including water, and bodily fluids from humans and animals, including those as described above. The absorbent collection article can be formed into numerous products used in the capture and absorption of animal or human fluids including sanitary articles such as medical or personal care collection devices. In examples, the absorbent collection article is configured to provide a urine collection device to be used in accommodating male incontinence or invalided immobility, or even prolonged periods of immobility such as space flight. In other examples, the absorbent collection article is configured to provide an article for collecting urine, absorbing urine, or both.

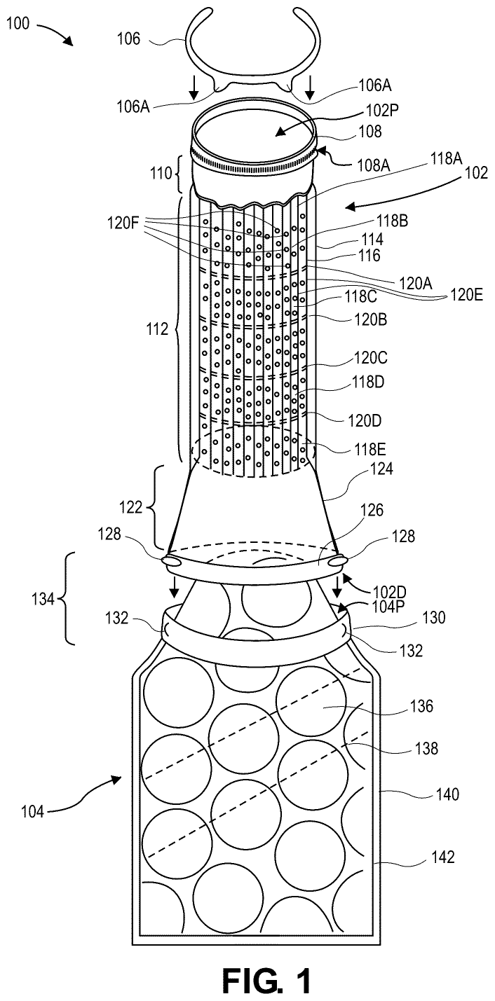

FIG. 1 is a schematic view of an aerating urine collection article, in accordance with the present disclosure. A urine collection article 100 including an aerator portion 102 having a proximal opening 102P and a distal opening 102D and a urine receptacle 104. A clasp 106, alternately referred to as a clamp, which in this view includes optional protrusions 106A located thereupon for manual opening by spreading open the w-shaped clasp is shown. It should be noted that w-shaped clamps, w-shaped clamps or other shapes may be used in alternate examples, provided they function as required in releasably holding the urine collection article onto a garment as described herein. In certain examples, the clasp may appear or be configured as a w-shaped clasp having with thumb or finger pads as protrusions. The clasp 106 is configured to attach the urine collection article 100 to a garment, in particular, an undergarment at a retention ring 108, also referred to as a notched ring, which is located at the proximal end 102P of the aerator 102 and includes a notch 108A or recession to receive the clasp 106 within the notch 108A. The proximal grooved ring 108 attaches the aerator portion 102 by means of the c-shaped clamp 106 onto an underwear garment. The retention ring 108 is attached to a first fabric layer 114 and a second fabric layer 116. The bi-layer of fabric comprised of the first, outer fabric layer 114 and a second, inner fabric layer 116 provides an upper flexible portion 110 or transition to an aerator tube or channel 112 at the proximal end 102P of the aerator 102. The channel 112 may be a segmented channel 112 as shown in FIG. 1, having from about one to about five segments, and as many as ten segments. The channel 112, in exemplary examples, includes a plurality of perforations 120F, may be fabricated from a corrugated material or have a combination thereof. The perforations 120F can be in a variety of patterns or spatial arrangements around a circumference of the channel 112 and provide ventilation and acration from outside the channel 112 to the inside of the channel 112. The corrugations 120E in the material of the channel 112 can be oriented in one or more directions, such as diagonal, horizontal, vertical, or a combination thereof. Rather than corrugations 120E, the tube may be created with a series of longitudinal or horizontal folds, likewise perforated and encased in NWNP fabric. The material comprising the channel can be paper, cardboard, or other biodegradable or compostable material. Other suitable materials can include wax-embedded paper which can improve conformation of corrugations 120E and limit absorption by paper at any perforations 120F. The thickness of the paper or channel material can range in thickness from about 0.05 to about 0.6 mm. In certain examples, the channel material, paper, is corrugated and joined to form a 10-14 cm circumference, at the inner corrugations and having a 8-12 cm long tube or channel which is perforated with 0.01-3.0 mm holes. Perforations 120F are placed to optimize breathability and strength and water resistance. Perforation of the paper is accomplished during the manufacture of the paper and its corrugation. Corrugations 120E run lengthwise and can number 10-20 in the circumference of the channel 112 and provide improved structural integrity. Paper and corrugations 120E and perforations 120F are made to optimize expandability, strength and lightness of the device 100. It should be noted that while biodegradable or compostable materials may be described herein, these categorizations are non-limiting, and that other examples may include non-biodegradable materials.

Also formed by the first, outer fabric layer 114 and second, inner fabric layer 116 is a lower flexible portion 122 or transition at a distal end 102D of the aerator 102. It should be further noted that the aerator channel 112 includes a circumferential wall, or alternatively an outside wall of a rectangular or triangular or otherwise polygonal channel, that is enclosed on either side of the channel wall and held in its shaped configuration by the first, outer fabric layer 114 and the second, inner fabric layer 116. The first, outer fabric layer 114 and second, inner fabric layer 116 can be adhered to the respective walls of the aerator 112 using an adhesive. The first fabric layer covers an inner circumference of the channel, while a second fabric layer covers an outer circumference of the channel. The fabric can be a non-woven, needle-perforated bamboo (or other NWNP fabric) which is adherent to the inner and outer surface of the corrugated tube forming the aerator channel 112. This fabric is water-resistant and breathable. The water resistance or hydrophobicity of the fabric is such that it does not absorb or allow flow through but sheets water with minimal gravity and is still breathable. Again, the fabrics 114, 116 are joined at the proximal end 102P of the aerator 102 and as it is thus doubled is extended 0.5-(1.5)-(2.0)4.0 cm and then attached to the retention ring 108 grooved with the notch 108A on its outer circumference. When the inner and outer NWNP fabrics 114, 116 are conjoined at the distal end 102D of the aerator 102 channel they transition from round to very flat oval over a distance of about 4 cm, but can be in a range from about 2 to about 7 cm, which can be accomplished without having to flare the fabric. This aspect is important in manufacturing the device 100 as it can serve to simplify the production.

As further illustrated in FIG. 1, the aerator 112 is segmented, including a first segment 118A, a second segment 118B, a third segment 118C, a fourth segment 118D, and a fifth segment 118E. The segments 118A, 118B, 118C. 118D, and 118E are separated by one or more flexible joints 120A, 120B, 120C, and 120D interspersed between each of the segments 118A, 118B, 118C. 118D, 118E and comprised of the first, outer fabric layer 114 and second, inner fabric layer 116. The aerator portion 102 provides a water resistant, breathable material encasing a corrugated paper tube that allows aeration for a penis that is placed therewithin. This aeration limits infection, especially fungus or yeast which are promoted by a moist environment and provides greater comfort for the user. These and other issues are problems associated with existing alternatives such as, but not limited to diapers and condom type catheters. The corrugations and segments present in the aerator 112 provide additional points of ventilation and allow for dilation to accommodate penile erections yet provide reduced bulk and lightweight strength in the penile resting state. Towards a distal opening 102D of the aerator portion 102, is the lower flexible portion 122 or transition (distal), comprised of a dual layer 124 of the first, outer fabric layer 114 and second, inner fabric layer 116, which terminates in a aerator pliable flat oval ring 126, which can be considered a first connection ring at a distal end of the aerator 102 which serves to hold the ends of the first, outer fabric layer 114 and second, inner fabric layer 116 layers together and join the aerator portion 102 to the urine receptacle 104 at a corresponding receptacle pliable flat oval ring 130, which can be considered a second connection ring. The first connection ring 126 includes one or more prominences 128 on an outer circumference. The second connection ring 130 is located at a proximal end 104P of the receptacle 104 and is fixedly attached to the receptacle. The second connection ring 130 includes one or more recesses 132 on an inner circumference. The aerator pliable ring 126 includes one or more prominences 128 or protrusions which correspond to and reversibly fixedly attach to one or more grooves 132 on an inner circumference of the receptacle pliable ring 130 (which can be considered a second connection ring 130 at a proximal end 104P of the receptacle 104), resulting in a set of locking rings 134 that interlock to fixedly attach the aerator portion 102 to the urine receptacle 104 portion. Oval ring dimensions can be defined by a diameter and circumference mean of the corrugations and provide adequate width to provide passage for a stockinette or absorbent proximal end of the receptacle 104 to achieve contact for good flow. Distal to the second connection ring 130 or receptacle oval ring the receptacle 104 may alternatively flare out into a waterproof receptacle. As the oval ring attachment or set of locking rings 134 will be frequently used, the rings can be designed to be user friendly and foolproof. The proximal oval ring will be depressed each time the receptacle is replaced. It may further include thumb or finger pads for depression, and each ring should have a nominal depth of approximately 1 cm. Certain examples of the urine collection article 100 can include a reversed arrangement, such as the second connection ring 130 fitting over the first connection ring 126, for example or can include other means of connecting the aerator 112 to the receptacle 104. At the distal end 102D of the aerator 102 the fabrics 114, 116 are joined and expanded, by addition of a NWNP fabric, into a flat oval bell comprising the lower flexible portion 122 for an approximate length of 1 to 4 cm in length terminating at the aerator pliable ring 126 on the inside of a 1 cm wide flat oval ring 1-(2)-3 cm×3-(6)-8 cm. The flat oval bell comprising the lower flexible portion 122 is a waterproof bridging fabric attached to the distal end 102D of the corrugated tube aerator 112 and extends into the aerator pliable ring 126 of the aerator 112. This bridge adds durability, provides reusability, and prevents any leakage during wearing and use. The fabric as described herein can be adherent to the paper aerator 112 on the inside and outside. The fabric is doubled and jointed in 4 points along the tube aerator 112 in some examples. The segmented portion of the tube aerator 112 can have spaces therebetween with about 1 mm-3 mm, in a nominal case, 2 mm, between the segments, providing improved flexibility and comfort. The aerator pliable ring 126 inserts and snaps onto a similar but slightly larger flat oval ring, the receptacle pliable ring 130 which is attached to the disposable pouch, or urine receptacle 104. The oval interlocking rings 134 are constructed of springy material, the smaller with thumb-pads projecting proximally and will snap together by prominences on the rings as described. The aerator pliable ring 126 or the receptacle pliable ring 130 which form the interlocking rings 134 may be formed out of cardboard, 3-D printed out of plastic, cast, or forged out of metal. In certain examples, there may be waterproof compostable fabric which bridges between the channel and the proximal flat oval ring and is outside the conjoined non-woven needle perforated fabric.

Absorbent media 136 such as cotton, bamboo, hemp, jute, wheat, cane grass, wool, and other natural seed, leaf, fruit, or stalk fibers, such as other compostable non-biological absorbent materials having superior capacity which absorbs any bodily fluids introduced into the aerator 102 that ultimately deposit into the urine receptacle 104. Waterproof or liquid proof material also forms an outer layer 140 of the urine receptacle 104. A stockinette layer 142 is a fabric material enclosure 142 which encapsulates and contains the absorbent media 136 and is held within the urine receptacle 104. The fabric material enclosure may be composed of a compostable or biodegradable material such as the aforementioned non-woven or needle-penetrated fabrics used in other portions of the urine collection device 100. The stockinette layer 142 may optionally include stitching 138 to stabilize or hold the absorbent media 136 in place within the stockinette layer 142. The absorbent material 136 can be removable from the receptacle 104 after use or discarded after use with the receptacle 104. In certain examples, fragrance may be added to one or more components of the urine collection device 100, as people experiencing incontinence are not always aware of incontinence related odors. Illustrative examples of applicable fragrances include essential oils, natural oils or other fragrances that are biodegradable or compostable. In examples of the present disclosure, when the urine collection device 100 is in use, urine sheets off the non-woven fabric when in contact within the aerator 112 or other portions of the fabric and falls into the stockinette layer 142 and absorbent media 136 where gravity and capillary action of the stockinette layer 142 and absorbent media 136 draw the urine into the absorbing bag-like stockinette layer 142 within the receptacle 104. In some examples, the fabric may be treated with a wax or biodegradable polymer to further enhance water-resistant or liquid sheeting properties. The fabric 114, 116 coating may further act as an adherent to other components or layers within the urine collection device 100.

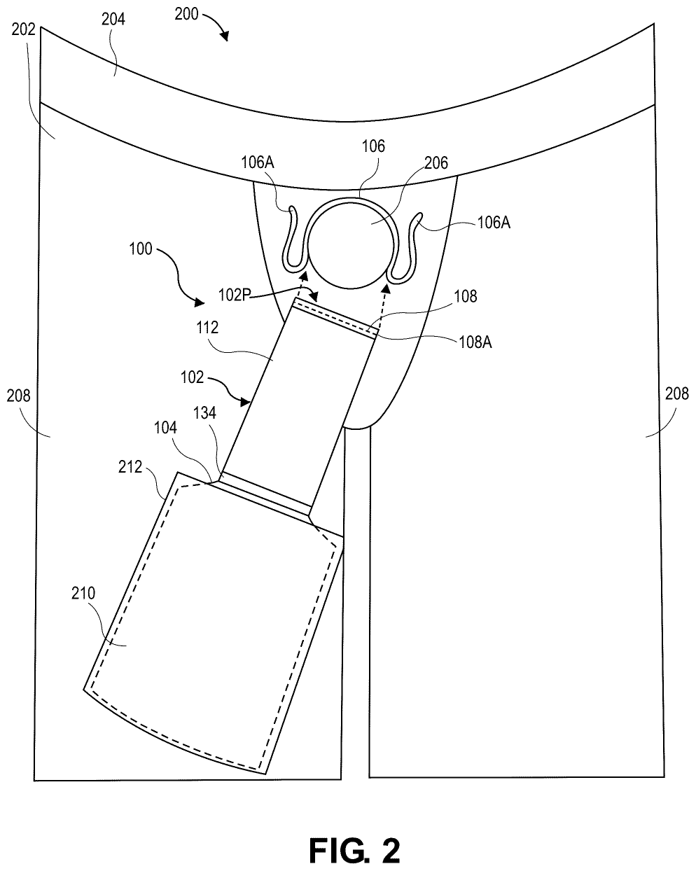

In certain examples, the urine receptacle 104 is a separable pouch intended to be discarded when full. A further aspect includes the formation of a protrusion of absorbent media and the stockinette layer 142 forming a 2 cm protrusion filled with absorbent media 136, which will extend into the interlocking rings 134 portion of the aerator 112 such that it is in contact with the joined NWNP fabric 114, 116. From this area the stockinette 142 is drawn into the receptacle 104, which in some examples can be a rectangular bag of dimensions to hold from about 100 mL to about 500 mL, or from about 150 mL to about 250 mL, or from about 300 mL to about 500 mL of urine. This stockinette 142 bag, loosely filled with absorbent media 136 such as cotton, cotton fluff, bamboo, bamboo fluff, or other materials described herein, which is evenly distributed by threads drawn throughout. This stockinette 142 bag is encased in a compostable waterproof bag or receptacle 104 attached to the distal end of the aerator 112. The waterproof casing provides additional structure to the receptacle 104. The receiving ring of the receptacle 104, or the second connection ring 130 is outside (larger than the first connection ring 126) is larger such that it serves to prevent drippage during use. When used in combination with a corresponding undergarment, the urine collection device 100 may be considered as a part of a urine collection apparatus, as illustrated in FIG. 2.

FIG. 2 is a schematic view of an aerating urine collection apparatus, in accordance with the present disclosure. An aerating urine collection apparatus 200 includes an undergarment 202 having a waistband 204 and an opening 206 providing access to the penis via the undergarment 202. The undergarment 202 is in the form of low-rise type underwear, fashioned to sit beneath the iliac crest in order to accommodate various sized anatomies, including obese users or users with a redundant abdomen. The underwear 202 also allows space for the scrotum to be contained. The undergarment includes two legs 208, one of which further includes a sewed in sleeve, also referred to as a pouch or pocket 212 for retaining, holding, stabilizing, and supporting the weight of the detachable urine receptacle 104. Underwear has legging of reasonable length with a sleeve to accept the rectangular pouch. The sleeve in the underwear holds the pouch. Legging has a sleeve built in to the medial-anterior aspect of the right legging and comfortably but assuring gravity (when person is lying down) accepts the pouch. The sleeve is of similar dimensions as the rectangular pouches. The sleeve extends to a 150 mL to 250 ml size where the distal end is interrupted by a zipper which when open allows the 300 mL to 500 mL pouch to extend the full length of the sleeve. In other examples, the size of the sleeve would accommodate other sizes of the receptacle 104. For ease of insertion of the bags into the sleeve there is a cuff at the end of the bag so the hand may draw the bag into the sleeve. Note that underwear will come in various sizes both to accommodate waist and buttocks but also thigh size so that placement and security of sleeve is maintained. A spring ring clasp 106, which in this view is w-shaped having optional protrusions 106A located thereupon for manual opening by spreading open the w-shaped clasp 106 is shown. The clasp 106 is affixed at the opening 206 of the undergarment 202 and configured to reversibly attach a urine collection article 100 having an aerator 112 to the undergarment 202, in particular, the undergarment 202 at a retention ring 108, also referred to as a notched ring, which is located at the proximal end 102P of the aerator 102 portion and includes a notch 108A or recession to receive the clasp 106 within the notch 108A. As described in regard to FIG. 1, an interlocking ring 134 reversibly attaches and connects the aerator 112 to the urine receptacle 104. Clamps, clasps, or interlocking rings may require a spring quality, and therefore can be comprised of metal, such as rustable steel as to render these components biodegradable and more easily broken down or recycled after use.

There is intent for this device and the underwear to be fabricated from 95%+biodegradable and compostable content. In certain examples, the aerator device may be fitted with a longitudinal rip string or similar mechanism such that the aerator can easily break away if trapping an engorged penis. Such a rip string for removal would be sealed longitudinally along the aerator in a fashion proximal to distal that allows for a lengthwise removal as needed. It is envisioned that disposable, biodegradable, compostable reservoir pouches can be sold in bulk. The devices and apparatus of the present disclosure provide better hygiene and ecologically sound alternatives that can be inexpensive to manufacture, inexpensive to buy, with reduced waste entering the waste stream or landfill. The aerator portion of the urine collection device can be sold separately, and in some cases, can last for many reuses if carefully maintained. It is closely estimated that the dimensions stated will accommodate all but about 5% of male penises, including those in a non-erotic erect state. Special orders may be placed for needs outside these dimensions. The rings at the top and at the bottom can be included in the manufacturing of the urine collection device. A flare at the end may be needed because the corrugations are no longer “taking in” the fabric at that location on the device. The detachable, reusable aerator device can be rinsed lightly, letting water run through it but not soaking or soaping which would in time saturate and weaken the corrugated tube or in some instances, a sterilizing medium may be provided for effective cleansing and sanitation. A metal drying frame can be purchased separately, in a wall mounted arrangement such that the frames are horizontal, or there is a similar rack which can stand on countertop or in a drawer.

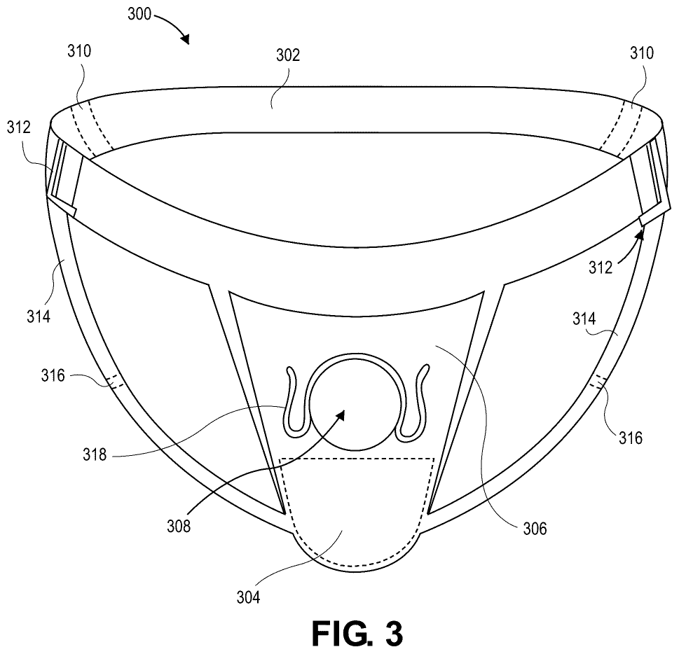

FIG. 3 is a schematic view of an aerating urine collection apparatus, in accordance with the present disclosure. In situations involving bedridden or otherwise invalided persons, instead of jockey type shorts, a modified classic jock strap can be provided. As illustrated in FIG. 3, a modified jock strap 300 is shown, where modification includes where an elastic waist belt 302 can be small, medium, large, or extra-large in size, and is low rise and is fit to ride below the iliac crests. The elastic waist belt 302 includes one or more adjustment points 310, which can be made from hook and loop fabric attachments, snaps, or other suitable adjustment mechanisms. At a front center is affixed a w-clasp 318 which is open at the bottom such that the aerator over penis device can snap into the undergarment through an opening 308 in a front panel 306. Leg straps 314 of 2-4 cm width are affixed to the elastic waist belt 302 and run under each buttock and pass through a buckle 312 on either side of the elastic waist belt 302 attached to the inferior aspect of the waist belt 302 in line with the anterior iliac crests. Buckles as described herein may be fabricated from fibrous materials, that are cleanable and compostable. Illustrative examples can include metal or plastic that is cast, 3D printed, or can include a combination thereof. These leg straps 314 can be adjusted by or secured by hook and latch fastening strips 316. There is further provided with such a system a scrotal shield 304 as part of the front panel 306 which can also be secured by hook and latch fastening strips to the legs straps 314 at their anterior origin. This scrotal shield 304 would be inexpensive and may be discarded if contaminated by feces or other bodily fluids. The front panel 306 can further include a fastener of hook and latch material, not shown here, configured to hold and secure a receptacle as described herein. The elastic belt 302 can be adjustable and available in a range of sizes to accommodate various anatomies. In exceptional cases, special underwear can be sold separately and individually tailored as a special order. Certain examples may incorporate a universal adjustable belt with an aerator adapter and disposable scrotal shield available for bed ridden patients. Elastic waistbands as described may alternatively be biodegradable, and thus can include plant-based or latex-based materials. While some users may experience a localized allergic reaction to latex-based materials, this should result in discontinued use. Alternative materials may be used in such cases. In still other examples, the waistband or leg straps may be constructed of adjustable materials including, for example, hook and latch material or buckles or a combination thereof, and can also be non-elastic.

It should be noted that the devices, articles, apparatus, and components thereof described in the present teachings may be fabricated from, but are not limited to, at least a portion with compositions including home compostable or plant-based plastics, in addition to other specific materials or categories of materials mentioned previously. It should further be noted that non-biodegradable materials as necessitated by the availability or non-availability of such materials may be used, and removed from devices, articles, apparatus, and components thereof prior to composting or recycling.

While the present teachings have been illustrated with respect to one or more implementations, alterations and/or modifications may be made to the illustrated examples without departing from the spirit and scope of the appended claims. For example, it may be appreciated that while the process is described as a series of acts or events, the present teachings are not limited by the ordering of such acts or events. Some acts may occur in different orders and/or concurrently with other acts or events apart from those described herein. Also, not all process stages may be required to implement a methodology in accordance with one or more aspects or embodiments of the present teachings. It may be appreciated that structural objects and/or processing stages may be added, or existing structural objects and/or processing stages may be removed or modified. Further, one or more of the acts depicted herein may be carried out in one or more separate acts and/or phases. Furthermore, to the extent that the terms “including,” “includes,” “having,” “has,” “with,” or variants thereof are used in either the detailed description and the claims, such terms are intended to be inclusive in a manner similar to the term “comprising.” The term “at least one of” is used to mean one or more of the listed items may be selected. Further, in the discussion and claims herein, the term “on” used with respect to two materials, one “on” the other, means at least some contact between the materials, while “over” means the materials are in proximity, but possibly with one or more additional intervening materials such that contact is possible but not required. Neither “on” nor “over” implies any directionality as used herein. The term “conformal” describes a coating material in which angles of the underlying material are preserved by the conformal material. The term “about” indicates that the value listed may be somewhat altered, as long as the alteration does not result in nonconformance of the process or structure to the illustrated embodiment. The terms “couple,” “coupled,” “connect,” “connection,” “connected,” “in connection with,” and “connecting” refer to “in direct connection with” or “in connection with via one or more intermediate elements or members.” Finally, the terms “exemplary” or “illustrative” indicate the description is used as an example, rather than implying that it is an ideal. Other embodiments of the present teachings may be apparent to those skilled in the art from consideration of the specification and practice of the disclosure herein. It is intended that the specification and examples be considered as exemplary only, with a true scope and spirit of the present teachings being indicated by the following claims.

What is claimed is:

1. A urine collection device, comprising:
an aerator, comprising:
a channel comprising paper;
a first fabric layer covering an inner circumference of the channel;
a second fabric layer covering an outer circumference of the channel;
a retention ring at a proximal end of the aerator, attached to the first fabric layer and the second fabric layer; and
a first connection ring at a distal end of the aerator, attached to the first fabric layer and the second fabric layer; and
a receptacle, comprising:
a second connection ring at a proximal end of the receptacle, attached to the receptacle; and
an absorbent media held within the receptacle.

2. The urine collection device of claim 1, wherein the retention ring is configured to be reversibly fastened to a garment.

3. The urine collection device of claim 1, wherein the first connection ring and the second connection ring are configured to reversibly fasten the aerator to the receptacle.

4. The urine collection device of claim 1, wherein the first connection ring comprises one or more prominences on an outer circumference.

5. The urine collection device of claim 4, wherein the second connection ring comprises one or more recesses on an inner circumference.

6. The urine collection device of claim 5, wherein the one or more prominences on the inner circumference of the first connection ring correspond to and interlock with the one or more recesses on the inner circumference of the second connection ring to fasten the aerator to the receptacle.

7. The urine collection device of claim 1, wherein the receptacle comprises a liquid proof material.

8. The urine collection device of claim 1, wherein the absorbent media is removable along with the receptacle.

9. The urine collection device of claim 1, wherein the absorbent media comprises a biodegradable material, a fabric material enclosure, and a biodegradable fill material.

10. The urine collection device of claim 1, wherein the absorbent media comprises cotton.

11. The urine collection device of claim 1, wherein the channel comprises a segmented channel comprising more than one segment.

12. The urine collection device of claim 11, wherein the segmented channel comprises five segments.

13. The urine collection device of claim 1, wherein the channel comprises a plurality of perforations, a corrugated material or a combination thereof.

14. The urine collection device of claim 1, wherein the first fabric layer comprises a non-woven fabric, a needle penetrated fabric, or a combination thereof.

15. The urine collection device of claim 1, wherein the second fabric layer comprises a non-woven fabric, a needle penetrated fabric, or a combination thereof.

16. A urine collection apparatus, comprising:
a garment comprising an external coupling;
an aerator, comprising:
a channel comprising paper;
a first fabric layer covering an inner circumference of the channel;
a second fabric layer covering an outer circumference of the channel;
a retention ring at a proximal end of the aerator, attached to the first fabric layer and the second fabric layer; and
a first connection ring at a distal end of the aerator, attached to the first fabric layer and the second fabric layer; and
a receptacle, comprising:
a second connection ring at a proximal end of the receptacle, attached to the receptacle; and
an absorbent media held within the receptacle.

17. The urine collection apparatus of claim 16, wherein the retention ring is configured to be reversibly fastened to the external coupling on the garment.

18. The urine collection apparatus of claim 16, wherein the first connection ring and the second connection ring are configured to reversibly fasten the aerator to the receptacle.

19. The urine collection apparatus of claim 16, wherein the aerator further comprises a flexible portion at a distal end.

20. The urine collection apparatus of claim 16, wherein the aerator further comprises a flexible portion at a proximal end.

21. The urine collection apparatus of claim 16, wherein the garment comprises a pouch to temporarily retain at least a portion of the aerator or the receptacle.

22. The urine collection apparatus of claim 21, wherein the pouch is included in one or more leggings of the garment.

23. The urine collection apparatus of claim 21, wherein the pouch is internal to the garment.
